(19) United States
(12) Patent Application Publication
(10) Pub. No.: US 2007/0020222 A1
(43) Pub. Date: Jan. 25, 2007

(54) Environmental control of confined animal rearing facilities with liquid iron compounds

(75) Inventor: Christopher B. Lind, Flanders, NJ (US)

Correspondence Address: ARTHUR J. PLANTAMURA;GENERAL CHEMICAL PERFORMANCE PRODUCTS LLC., 90 EAST HALSEY ROAD, PARSIPPANY, NJ, 07054, US

(73) Assignee: General Chemical Performance Products LLC, Parsippany, NJ (US)

(21) Appl. No.: 11/495,208

(22) Filed: Jul. 28, 2006

Related U.S. Application Data
(63) Continuation-in-part of application No. 10/887,281, filed on Jul. 8, 2004, now abandoned.

(51) Int. Cl. A61L 9/00

(52) U.S. Cl. 424/76.2

(57) ABSTRACT

A system is provided for enhancing the environment in enclosed facilities for rearing livestock. The system involves the addition of a suitable amount of liquid ferric sulfate or ferric chloride a mixture thereof to bedding material or to the floor area where the livestock is confined. The concentration of the ferric compound in the applied liquid is sufficient to maintain a pH of about 7.5 and most preferably between 4.0 and 7.0 in the confined environment and such that it inhibits ammonia volatilization and stabilizes, i.e. insolubilizes any resulting soluble phosphorus generated.

### CROSS-REFERENCE TO RELATED APPLICATIONS

This application is a continuation-in-part of application Ser. No. 10/887,281 filed on Jul. 8, 2004.

### THE INVENTION

This invention relates to a system for enhancement of conditions prevailing in domestic animal (i.e. livestock) rearing facilities and to the control of environmental effects from the manure generated by such livestock at these facilities. More specifically, the present invention relates to the improvement of the atmospheric environment for animals during the rearing of livestock in substantially confined facilities. In particular, the invention deals with the problem of ammonia volatilization and odors and with soluble phosphorus runoff from manure in domestic animal rearing facilities. By using a treatment comprised of applying to the bedding or floor area where the animals are confined a liquid containing essentially ferric sulfate or ferric chloride or mixtures thereof.

Various building construction designs are known for housing and for sheltering livestock such as poultry, including chickens, turkeys, ducks, ostrich, swine and dairy cows in rearing facilities on farms. Conventional enclosure types, for example are such on the kind described in U.S. Pat. No. 5,890,454 comprising a flooring which includes a grate or slatted section to allow manure which accumulates to fall through the flooring opening into a manure collection pit. In such facilities the manure must be periodically cleansed, i.e. removed from the pit. Another type of facility includes an appropriately inclined floor, such as a concrete floor, which has at least one flushing trough or channel defined in the floor. Manure temporarily collects on a portion of the floor and will present a detrimental effect on the health and growth cycle of the livestock. While from time to time the area bedding is replaced and/or the animal rearing floor area is flushed, in the interim, the atmosphere created by animal manure deposits presents an unhealthy period. The invention by periodically applying liquid ferric compound treatment to alleviate the interim contamination forestalls the creation of an adverse environment. The invention contemplates the installation of a liquid dispersing system that periodically applies and thereby depresses the generation of ammonia and other adverse impacts such as soluble phosphorus formation, by applying the liquid ferric dispersion in cycles of every 8 to 12 hours, for example.

Usually these facilities are roofed and may be confined and use no absorbent bedding material permitting the manure to be relatively efficiently handled as a slurry or flowable liquid. The manure is diluted with water to the extent practical to assist in transporting and applying the manure.

In facilities of this kind, the accumulation of manure results in the production of ammonia, which accumulates and saturates the contiguous atmosphere. This ammonia is generated in relatively high quantities, and at more severe levels, is toxic to the animals as well as to humans present in substantially enclosed animal rearing spaces. Further, this ammonia reacts in the atmosphere to form particulate material (PM) of a size that is injurious to human health, PM _{2.5} and PM ₁₀. The subscripts denote particle size in micrometers.

A severe related problem arising from effluent produced from the animal rearing facilities of this kind is the generation of soluble phosphorus and hydrogen sulfide, which are generated by-products that are also of substantial environmental concern.

While the prior art, e.g., U.S. Pat. No. 5,622,697 discloses the use of iron compounds including ferric and ferrous sulfates and chloride, these compounds have heretofore been suggested as being of possible utility as solids. However, the use of such iron compounds as granular solids in the animal rearing facilities suffer from severe drawbacks in that the animals are likely to ingest solids and results in illness and is often fatal. Additionally, the use of the iron compound in solid form to be distributed in practical quantities is by its nature relatively only marginally effective.

The invention is also distinguishable from an organic waste treatment system in which ferric sulfate or ferric chloride is utilized as a precept and following the treatment of the waste with an oxidizing agent such as that described in U.S. Pat. No. 4,108,771. Such oxidizing agent precursors are regarded as hostile to the livestock.

The invention deals essentially with: 1) Control of ammonia (NH₃) and the odors resulting from ammonia, hydrogen sulfide (H₂S), volatile fatty acids (VFA) and the like resulting from the decomposition of animal manures within the growing facility and during the livestock growth cycle; 2) Reduction of soluble reactive phosphorus (SRP) concurrent with the odorous substance reduction. SRP is a pollutant in surface water; 3) Reductions in particulate matter emissions (PM_{2.5} and PM₁₀) concurrent with and generated by odorous substances most notably ammonia; 4) Reduction of the pH of the litter, bedding or manure to effect odor control; and 5) A mechanism to produce a sufficiently low pH and free water to inhibit proliferation of microbes and insects especially those regarded as pathogens.

To realize these objects the application during the livestock rearing cycle of a treatment that comprises essentially liquid ferric sulfate and/or ferric chloride to the litter, to the bedding or to the flooring on which the livestock leave manure deposits, is employed. Amounts of ferric sulfate vary with type of animal waste and its physical and chemical characteristics but typical applications comprise the use of about ten (10) to about thirty (30) gallons per thousand square feet of floor area surface on which the bedding or litter treatment is present or on which the livestock dwells. The treatment comprises a liquid containing from about 5% to about 15%, preferably about 8% - 13% and most preferable 10% - 12% ferric sulfate, as iron, in the liquid applied to the litter as flooring.

The advantage of the system of the invention over the various alternatives such as use of aluminum salts such as aluminum sulfate, aluminum chloride or aluminum nitrate, sodium bisulfate¹, clay mixed with acid (normally sulfuric), phosphoric acid, microbial preparations and enzymes, either in liquid or solid form, include the following:

1. Enzymes and microbial preparations function on the theory that the metabolic pathways of said waste decomposition can be changed to produce metabolites that are not odorous or toxic. Being biologicals they require specific growth environments and rarely demonstrate lasting effects without constant and costly reapplication. Further they do not reduce phosphorous and in fact may increase the soluble fraction through their own digestive processes thereby increasing the potentially detrimental environmental effects. Biologicals do not depress pH to control pathogens and insects rather they exhibit pH sensitivity and can die off if pH stressed. Liquid iron products are also more widely available at “commodity” pricing.

2. Phosphoric acid has been widely used in the poultry industry for acidification of the litter, floor, and bedding. Although phosphoric acid is effective, stringent phosphorous discharge and land application limits essentially prohibit this practice. Furthermore, phosphoric acid is far more costly and is a more hazardous acid than a liquid ferric sulfate product. It is also noteworthy that liquid ferric sulfate and ferric chloride products are certified for use in drinking water assuring purity that commercial acids may not provide.

3. Acid clay mixtures are more corrosive and hazardous than liquid ferric sulfate (LFS) and some like products. Being dry products, the corrosive dusts present worker exposure issues and equipment and facility corrosion issues absent in LFS and others. Acid clay mixtures do not reduce SRP and may actually increase SRP by dissolving particulate phosphorous in manure through low pH. Acid clay mixtures do not reduce litter or bedding moisture as LFS does.

4. Sodium bisulfate (SBS) is a deliquescent salt that absorbs moisture from the air. In this manner it adds moisture to the litter—a negative aspect since reduction of free water is an essential aspect of controlling pathogens in litter. Sodium bisulfate is a dry dusty material that, like acid clay mixtures corrodes buildings and equipment and exposes workers to corrosive aerosols. SBS does not reduce SRP as LFS does. SBS contains approximately 20% (19-23%) sodium. Sodium will stay in the bedding or manure and when used as fertilizer will increase the salinity of the soil and ground water —both areas of environmental and agricultural concern. LFS contains parts per million sodium and has negligible effect on salinity. Iron is a plant nutrient and LFS treated wastes will have this nutrient as an artifact. SBS has limited utility in other applications and is produced domestically by only one producer; thus availability and pricing can be a concern.

5. Dry aluminum salts have a tendency to be dusty. While not nearly as corrosive as SBS or acid clay dusts, they can be problematic. LFS et al is applied as a liquid by custom applicators and has no dust issues. LFS is more concentrated in terms of net acidity and metal content thus fewer gallons are needed reducing potential application time. The higher iron (vs. aluminum) content results in SRP reduction at lower applied product volumes. Aluminum makes a less soluble phosphate than iron—in many cases this is desired. Some farmers prefer the slightly more soluble iron phosphate end product so they can better use the manure for its nutrient value to crop plants. There is an apparent problem with build up of aluminum in soils that is perceived to precursor aluminum toxicity. USDA studies document the fallacy of the perception—but it still persists. Iron on the other hand is not burdened with such perceptions.

6. None of the above technologies are regarded as effective to chemically precipitate H₂S. Iron compounds on the other hand have been used for decades for precipitation of sulfides from municipal sewage, sludge and industrial wastes.

Accordingly, the need is apparent for means to alleviate the relatively toxic atmospheric in, and to manage a suitable disposal of environmentally undesirable effluent from, such facilities.

### SUMMARY OF THE INVENTION

In accordance with the invention, a novel system which employs a liquid preferably aqueous ferric sulfate or ferric chloride or mixtures thereof, preferably the ferric sulfate in concentrations at from about 5% to about 15% by weight, as iron, that deals with the environmentally undesirable conditions and effluent resulting from livestock domestic animal rearing facilities in particular, during the livestock growing cycle, is provided. The system of the invention involves the periodic application during the livestock growing cycle of a suitable amount of liquid ferric sulfate and/or ferric chloride to bedding material or to the livestock inhabiting floor area. The liquid iron containing compounds may be applied to a manure soiled or fresh litter or to a floor area where the livestock is housed. The concentration of the liquid iron compound contained in the liquid dispersant is controlled to maintain a pH environment at or below a pH of about 7.5, and most preferably between 4.0 and 7.0. Although where the liquid iron additive is applied to bedding, particularly already soiled bedding, a manure slurry, an amount is added depending on the accumulated quantities and maturity of the deposit containing bedding to preferably maintain the bedding at a concentration such that the iron content applied inhibits ammonia volatilization and stabilizes, i.e. insolubilizes, the resulting soluble phosphorus.

### DETAILED DESCRIPTION OF THE INVENTION

The invention among the various advantages comprising the use of liquid ferric sulfate, or ferric chloride includes use of a readily available form of iron that is relatively safe; its activating effect is more rapid; it acts to promptly initiate the desired hydrolysis; and, in the case of litter bedding application, acidulates the litter more quickly. Liquid ferric sulfate is commonly a product produced by the digestion of an iron source (hematite, magnetite, scrap iron) with sulfiric acid. LFS can also be processed waste from steel pickling or other ferrous material manufacturing and processing. Typically the liquid ferric sulfate will have 10%-12% by weight iron and from about 0.1% to about 5.0% free acidity. It will have a specific gravity of less than about 1.3% to about 1.6% gm/cc. LFS can also be produced by dissolving various dry or solid ferric sulfate or ferrous sulfate products in water and processing to the desired final product strength and characteristics. It can also be a by-product of pigment (TiO₂) manufacturing. Liquid ferric chloride can also be utilized alone, or in combination with ferric sulfate; ferric sulfate being the preferred treatment mechanism. Ferric chloride can be produced by digestion of an iron source in hydrochloric acid or wet chlorine streams. The larger volumes are by-products from TiO₂ manufacturing or steel/iron manufacturing and processing. It will typically contain 10-12% iron and <1->3% free acid. Waste Pickle Liquor (WPL) is a ferric/ferrous chloride/sulfite by product of iron and steel manufacturing and processing. It is variable in chemical assay and strength tending to low (<5%) iron content and high (>5%) acidity. It is often used as a starting material for other products herein such as ferrous chloride or ferrous sulfate.

Liquid ferric sulfate normally may be processed from a ferric sulfate manufacturing plant, or from industrial waste acid streams or through dissolving scrap iron in sulfuric acid by diluting or concentrating the desired strength. Substituting hydrochloric acid for the sulfuric acid in the previous manner produces ferric chloride and ferrous chloride materials also suitable. Ferric sulfate is also available as dry materials from iron and pigment manufacturers and when utilized can be solubilized in water to the appropriate concentration for use.

In essence the invention provides an environment enhancing system comprising applying a compound selected from liquid ferric sulfate, liquid ferric chloride and mixtures thereof in amounts effective to suitably treat the adverse environmental impact on livestock during the livestock growing cycle and its physical and chemical characteristics. A suitable liquid iron dispersant concentration and application comprises of from about ten (10) to thirty (30) gallons containing about 5% to about 15% by weight of iron per thousand square feet of floor area of bedding or litter surface in which the livestock such as, poultry, swine or cattle are housed.

The invention will be further described by reference to the following examples. However, the invention is not meant to be limited to the details described therein. In the examples, parts and percentages are by weight unless otherwise noted.

### EXAMPLE 1

Use of liquid ferric sulfate as a litter amendment for broiler chicken manure.

One hundred grams of one-year old used broiler litter was added to 900 ml deionized water and mixed 30 minutes using a six place Phipps and Bird model 7790-400 jar stirrer. The litter sample was obtained 03-08-04 from a commercial operation in Springdale, AR growing Cobb-Vantress birds six weeks. Five flocks of 20,000-30,000 birds were raised on this rice hull litter which was decaked (manure mats removed partially) and top-dressed (replenished) with fresh rice hulls after every flock. No litter amendments were used.

To each of six samples aluminum sulfate was added as a known effect at doses per liter of litter and water of 0 (control) Ig, 2.5g, 5g, 10g and 20g. The test data with LFS at 12.1% Fe were based on 0.5g/1.0g, 2.5g, 5g, 10g and 20g. The pH was determined on samples settled for one hour using an Orion Ionanalyzer Model EA920. Target pH is <6 for ammonia control. SRP was determined on a Hach Model 780 Spectrophotometer using the Phosver®3 method. SRP was determined on diluted samples of litter. Deionized and distilled reagent blanks were also run.

| Dose | | Alum | | 12% LFS |
|---|---|---|---|---|
| g/l | pH | SRP | PH | SRP |
| 0 | 8.06 | 110 | 8.06 | 110 |
| 0.5 | — | — | 7.93 | 60 |
| 1.0 | 7.70 | 76 | 7.70 | 46 |
| 2.5 | 7.41 | 35 | 6.81 | 45 |
| 5.0 | 6.88 | 16 | 5.81 | 34 |
| 10.0 | 5.72 | 16 | 4.15 | 10 |
| 20.0 | 4.55 | 16 | 3.22 | 27 |

At equivalent dosages in grams of product per liter of sample LFS produced a low pH. Alum still produced generally lower SRP values—but LFS produced the lowest SRP. LFS produced the desired results.

### EXAMPLE 2

Based on experimental data a commercial broiler house(s) will be treated with LFS at 0, 7.5 gallons/1000 square feet and 15 gallons/1000 square feet. This is equivalent to 100# dry alum/1000 square feet and 200# dry alum/1000 square feet or 25 and 50 gallons of 7% liquid acid alum respectively. Analytical data include ammonia, pH of litter, SRP of litter, bird growth statistics including feed conversion, mortality and condemnation rate, insect infestation and litter microbiology.

Although the invention has been described in terms of particular embodiments, blends of one or more of the various additives described herein can be used, and substitutes therefore, as will be known to those skilled in the art. Thus the invention is not meant to be limited to the details described herein, but only by the scope of the appended claims.

What is claimed:

1. A method for treating a domestic livestock rearing environment contaminated with odors from (NH₃) ammonia, hydrogen sulfide (H₂S), volatile fatty acids (VFA) resulting from the decomposition of animal manures during the livestock growing cycle within a substantially confined growing facility to reduce ammonia levels and soluble reactive phosphorus (SRP) concurrent with the odorous substance reduction and to reduce the pH of the litter, bedding or manure to effect the odor and produce a sufficiently low pH and free water to inhibit proliferation of microbes and insects, the comprising applying a compound selected from the group consisting of liquid ferric sulfate, liquid ferric chloride and mixtures thereof at periodic cycles and in amounts effective to suitably treat the type of animal waste and its physical and chemical characteristics and within limits of from about ten (10) to thirty (30) gallons containing about 5% to about 15% by weight of iron per thousand square feet of floor area of bedding or litter surface.

2. The method of claim 1 wherein the concentration of the ferric sulfate, ferric chloride or mixtures is from about 10% to about 12% as iron and <1% to 20% by weight in liquid to solid manures.

3. The method of claim 2 as applied to poultry litter.

4. The method of claim 3 as applied to the litter for poultry selected from chickens, turkeys and ducks.

5. The method of claim 2 as applied to swine.

6. The method of claim 2 as applied to dairy cows.

7. The method of claim 1 wherein the compound is ferric sulfate and is applied in concentrations sufficient to maintain the pH between about 4.0 and 7.0.

8. The method of claim 1 wherein the compound selected is ferric sulfate that comprises about 10% to about 12% by weight iron and a specific gravity less than about 1.3 to about 1.5.

9. The method of claim 8 wherein the applied liquid sulfate has a pH of between about 4.0 and 7.0.
